(19) United States
(12) Patent Application Publication
(10) Pub. No.: US 2014/0113829 A1
(43) Pub. Date: Apr. 24, 2014

(54) SYSTEMS AND METHODS OF SELECTING COMBINATORIAL COORDINATELY DYSREGULATED BIOMARKER SUBNETWORKS

(71) Applicants: Mehmet Koyuturk, Shaker Heights, OH (US); Mark Chance, Chagrin Falls, OH (US); Rod Nibbe, Anchorage, AK (US); Salim Akhter Chowdhury, Cleveland, OH (US)

(72) Inventors: Mehmet Koyuturk, Shaker Heights, OH (US); Mark Chance, Chagrin Falls, OH (US); Rod Nibbe, Anchorage, AK (US); Salim Akhter Chowdhury, Cleveland, OH (US)

(73) Assignee: CASE WESTERN RESERVE UNIVERSITY, Cleveland, OH (US)

(21) Appl. No.: 13/642,777

(22) PCT Filed: Apr. 22, 2011
(86) PCT No.: PCT/US2011/033527

§ 371 Date: Jun. 7, 2013

Related U.S. Application Data
(60) Provisional application No. 61/326,897, filed on Apr. 22, 2010.

(51) Int. Cl. G06F 19/18; C12Q 1/68

(52) U.S. Cl. CPC G06F 19/18; C12Q 1/6886
USPC 506/8; 506/39; 506/13; 435/6.14; 702/19

(57) ABSTRACT

Systems and methods of selecting combinatorial coordinately dysregulated biomarker subnetworks are provided. In one embodiment, a method comprises comparing the normalized gene expression data to a predetermined threshold to provide binary gene expression data associated with phenotype samples and control samples, analyzing subnetwork states of the binary gene expression data associated with phenotype samples and control samples to identify gene expression patterns that occur in phenotype samples and do not occur in control samples and identifying a subnetwork that provides gene expression patterns indicative of a sample being a phenotype sample.

### RELATED APPLICATION

This application claims priority from U.S. Provisional Application No. 61/326,897, filed Apr. 22, 2010, the subject matter of which are incorporated herein by reference in their entirety.

### FIELD OF THE INVENTION

This application relates to functional genomics and proteomics, and particularly relates to systems and methods of selecting combinatorial coordinately dysregulated biomarker subnetworks.

### BACKGROUND

Functional genomics and proteomics analysis are important technologies for biomedical research in the post-genomics era. Expression proteomics, which explores the changes in gene or protein expression levels, is one of the most important aspects of proteomics research. The importance of these technologies is to understand the fundamental biology of development and disease as well as discover biomarkers for ascertaining disease diagnosis and prognosis. There are a number of well established technologies for quantitative analysis of proteomes; these include 2-dimensional differential in gel electrophoresis (2D-DIGE) with quantification by fluorescence analysis of labeled proteins and “shotgun” proteomics methods where quantification is performed using differential isotopic labeling of digested protein samples. The 2D-DIGE method of separation and quantification at the protein level is termed “top-down” proteomics, since the quantification is carried out at the intact protein level, while initial digestion followed by separation and quantification at the peptide level is termed a “bottom-up” approach. Both these experimental designs rely on the relative quantification of proteins within a control versus an experimental sample.

While 2D-DIGE is very useful in quantifying functional proteins, it can only screen a limited fraction of the proteins in a sample at a time. DNA microarray technology, on the other hand, enables screening of thousands of mRNA transcripts that are used to synthesize proteins. The mRNA concentration via microarrays, often called “gene expression” serves as a proxy to the amount of functional proteins in a sample.

A critical problem in understanding the role of specific genes in complex diseases is that the genes and their protein products interact in a dynamic fashion, such that an individual patient with a specific genetic background may respond to environmental factors in a unique leading to variable disease outcome. The utilization of combination of genes to provide diagnosis, analyze disease progression, or predict response to therapy has been a logical response as it has been thought that multiple variables may provide greater classification power. These genes were initially picked as biomarkers by statistical association or clustering methods, and in many cases provided ‘panels’ of up to hundreds of genes that could be employed to classify patients.

### SUMMARY

An aspect of the application relates to a system of selecting combinatorial coordinately dysregulated biomarker subnetworks. The system can include memory for storing computer executable instructions and a processor for accessing memory and executing computer executable instructions. The computer executable instructions include a gene data binarization component that compares continuous scored gene expression data associated with phenotype samples and control samples to a predetermined threshold to provide binary gene expression data associated with phenotype samples and control samples and a combinatorial search engine that analyzes subnetwork states of binary gene expression data associated with phenotype samples and control samples to identify gene expression patterns that occur in phenotype samples and do not occur in control samples to identify a subnetwork that provides gene expression patterns indicative of a sample being a phenotype sample.

Another aspect of the application relates to a method of selecting combinatorial coordinately dysregulated biomarker subnetworks. The method includes comparing normalized gene expression data to a predetermined threshold to provide binary gene expression data associated with phenotype samples and control samples, analyzing subnetwork states of the binary gene expression data associated with phenotype samples and control samples to identify gene expression patterns that occur in phenotype samples and do not occur in control samples and identifying a subnetwork that provides gene expression patterns indicative of a sample being a phenotype sample.

A further aspect of the application relates to combinatorial coordinately dysregulated biomarker subnetwork for determining metastasis of colorectal cancer (CRC). The combinatorial coordinately dysregulated biomarker subnetwork can include the gene expression profiles of at least two of TNFSF11, MMP1, BCAN, MMP2, thrombospondin 1 (TBSH1), or (osteopontin) SPP1. In an aspect of the application, the state function of TNFSF11, MMP1, BCAN, MMP2, TBSH1, and SPP1 indicative of metastasis of CRC are, respectively, low gene expression (L), low gene expression (L), low gene expression (L), low gene expression (L), and high gene expression (H) (i.e., LLLLLH).

Another aspect of the application relates to the use of expression profiles of the marker proteins listed above in a method for diagnosing an increased risk of development of metastatic colorectal cancer (CRC) in a subject. The method includes: (1) obtaining a biological sample from a subject comprising colorectal cancer cells; and (2) determining, in the cancer cells, the gene expression level of at least two selected from the selected from the group consisting of TNFSF11, MMP1, BCAN, MMP2, TBSH1, and SPP1, wherein a low level of TNFSF11, MMP1, BCAN, MMP2, and/or TBSH1 and/or high level of SPP1 is indicative of the cancer cells having an increased risk of being metastatic and the subject having metastatic colorectal cancer.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a block diagram of a system for selecting combinatorial coordinately dysregulated biomarker subnetworks in accordance with an aspect of the present invention.

FIG. 2 illustrates a phenotype gene expression matrix in accordance with an aspect of the invention.

FIG. 3 illustrates a control gene expression matrix in accordance with an aspect of the invention.

FIG. 4 illustrates a methodology for selecting a combinatorial coordinately dysregulated biomarker subnetwork in accordance with an aspect of the invention.

FIG. 5 illustrates a computer system that can be employed to implement systems and methods in accordance with one or more aspects of the invention.

FIG. 6 illustrates graphs comparing classification performance of subnetworks identified by CRANE in predicting colon cancer metastasis as compared to single gene markers and subnetworks identified by algorithms that aim to maximize additive coordinate dysregulation.

### DETAILED DESCRIPTION

The present invention relates to systems and methods of selecting combinatorial coordinately dysregulated biomarker subnetworks. The system and methods employ combinatorial coordinate dysregulation to analyze subnetwork states of a plurality of samples to determine specific gene expression patterns that provide an indication of whether a given sample is indicative of a phenotype (e.g., a disease) and/or indicative of not being a phenotype (e.g., normal). The systems and methods can be employed for a variety of biological conditions.

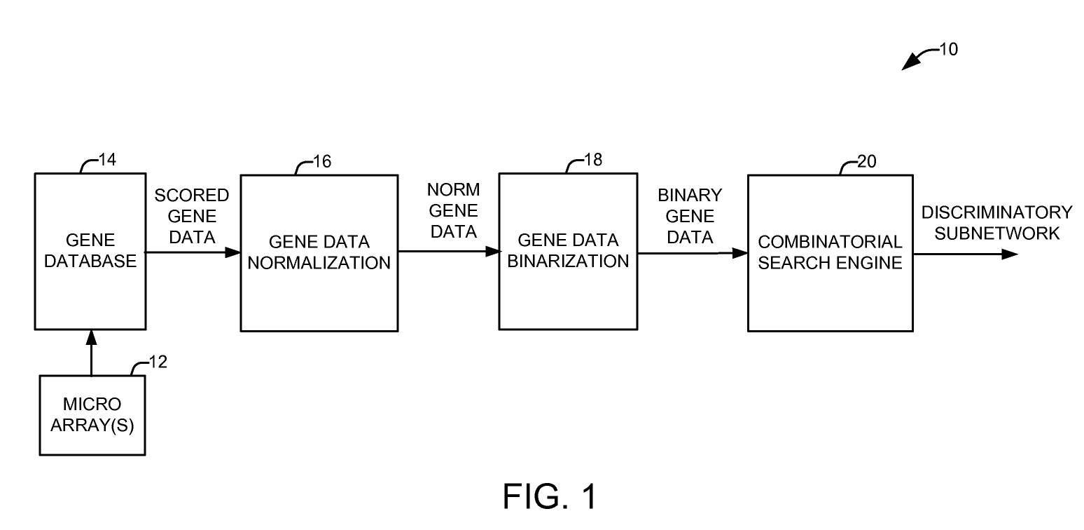

FIG. 1 illustrates a system 10 for selecting combinatorial coordinately dysregulated biomarker subnetworks in accordance with an aspect of the application. The system 10 includes a gene database 14 that is developed employing a plurality of microarrays 12 to provide a plurality of continuously scored gene phenotype samples and a plurality of scored gene control samples. The database 14 can be developed or obtained from a variety of public or commercial off the shelf (COTS) gene expression databases, such as for example, the gene expression omnibus database provided by the National Center for Biotechnology Information (NCBI). Gene expression databases provide gene expression data associated with genes that provide some correlation between one another for a given phenotype, such that the genes associated with the expression data can be characterized in the form of subnetworks. Each gene in a sample is provided with a continuous numerical score based on an amount of mRNA transcript present in the gene for the given sample. The scored gene data is provided to a gene data normalization component 16 that normalizes the scored gene data to remove the effects of experimental bias.

In one aspect of the application, the gene data is in the form of a gene expression matrix in which samples are assigned columns and gene expressions are assigned rows, such that a gene expression score value is assigned to a given gene expression associated with a given sample. The score values of each gene expression is divided by the median value of that column and then the mean of each row is subtracted from the value of each gene expression in the row and then divided by the standard deviation of that row. It is to be appreciated that this is one example of normalization of the scored gene data and that a variety of other normalization techniques could be employed to normalize the scored gene data.

The normalized gene data can then be provided to a gene data binarization component 18 that assigns a binary expression value to each scored gene expression value. The binary expression value can be assigned by comparing the normalized scored gene expression data to a threshold and assigning a binary logic ‘1’ to the expression data (e.g., high level of expression) if it is above the threshold and a binary logic ‘0’ if it is at or below the threshold (e.g., low level of expression). In this manner a given gene associated with a given sample is either expressed with a binary logic ‘1’ or not expressed with a binary logic ‘0’. The binary gene expression data is then provided to a combinatorial search engine 20. The combinatorial search engine 20 performs an exhaustive search on the binary gene expression data to identify subnetwork biomarkers by analyzing subnetwork states of a plurality of phenotype and control samples to determine specific gene expression patterns that provide an indication of whether a given sample is indicative of a phenotype (e.g., diseased sample) and/or indicative of not being a phenotype (e.g., normal sample). The combinatorial search engine 20 then generates a discriminatory biomarker subnetwork that can be employed to perform classification on unknown samples.

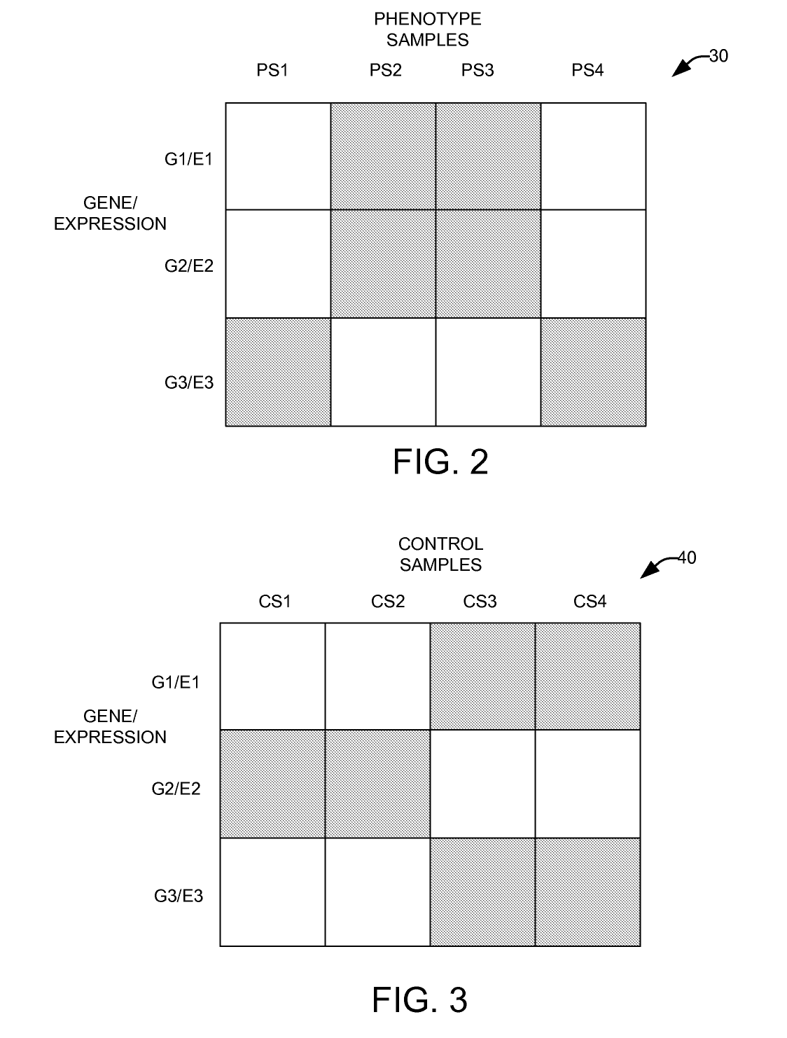

FIG. 2 illustrates a phenotype gene expression matrix 30 in accordance with an aspect of the application. The phenotype expression matrix 30 illustrates rows of gene/expressions G1/E1-G3/E3 for columns of phenotype samples PS1-PS4. Genes G1-G3 associated with gene expression values E1-E3 form a gene subnetwork. Each row is associated with a given gene with an expression value having a logic ‘0’ if not shaded and a logic ‘1’ if shaded.

FIG. 3 illustrates a control gene expression matrix 40 in accordance with an aspect of the application. Similarly, the control gene expression matrix 40 illustrates rows of the same genes/expressions G1/E1-G3/E3 for columns of control samples CS1-CS4. As can be seen in FIG. 2, the gene expression pattern of phenotype sample PS1 and phenotype sample PS4 is ‘001’ and the gene expression pattern of phenotype samples PS2 and PS3 is ‘110’. As can be seen in FIG. 3, the gene expression pattern of control sample CS1 and control sample CS2 is ‘010’ and the gene expression pattern of control samples PS3 and PS4 is ‘101’.

Therefore, phenotype samples exhibit a gene expression of either ‘001’ or ‘110’ and not a pattern of ‘010’ or ‘101’ and control samples exhibit a gene expression pattern of ‘010’ or ‘101’ and not a pattern of ‘001’ or ‘110’. Since gene expression patterns found in phenotype samples are not found in control samples, the gene subnetwork of G1-G3 provides for a discriminatory subnetwork of biomarkers for identifying a phenotype in an unknown sample and since gene expression patterns found in control samples are not found in phenotype samples, the gene subnetwork of G1-G3 provide for a discriminatory subnetwork of biomarkers for identifying an unknown sample not being a phenotype. Additionally, since both the above conditions are true, the gene subnetwork of G1-G3 provides for a discriminatory subnetwork of biomarkers for discriminating between phenotype and control samples.

As stated above with respect to FIG. 1, the combinatorial search engine 20 performs an exhaustive search on the binary gene expression data to identify subnetwork biomarkers by analyzing subnetwork states of a plurality of phenotype and control samples to determine specific gene expression patterns that provide an indication of whether a given sample is indicative of a phenotype (e.g., a disease) and/or indicative of not being a phenotype (e.g., normal). For example, for the three gene example of FIGS. 2-3, the combinatorial search engine 20 would first look at gene expression patterns of G1, G2, and G3 separately, then look at gene expression G1 and G2, G1 and G3, and G2 and G3, and finally look at gene expression patterns of G1, G2, and G3. It is to be appreciated that the search algorithm is enumerative such that the search patterns reviewed are each possible pattern of 1, 2, 3, 4 . . . N, such that N is an integer selected to maintain a reasonable computation time frame. However, the search engine does not explicitly enumerate all possible combinations, but rather prunes out chunks of the search space based on the mathematical framework described below.

The combinatorial search engine 20 quantifies by employing the mutual information between the gene expression patterns and phenotype as follows:

Let Êi denote the binarized expression profile of gene gᵢ and that gene gᵢ has high expression in sample sⱼ if Êᵢ(j)=1 and low expression if Êᵢ(j)=0. Then, the combinatorial coordinate dysregulation of subnetwork S is defined as:

I(F_{S};C)=H(C)−H(ClÊ₁, Ê₂, Êₘ) EQ. 1

where F_{S}={Ê₁, Ê₂, . . . , Êₘ}∈{0,1}^{m} is the random variable that represents the combination of binary expression states of the genes in S and m=|S|, H(C) is the Shannon entropy of an arbitrary sample being a phenotype before reviewing gene expression data, H(ClÊ₁, Ê₂, . . . , Êₘ) is the conditional entropy of the sample being a phenotype after reviewing gene expression data (measure of how much information gained by looking at the gene expression state of the subnetwork) and I(F_{S};C) is a measure that is maximized with the subnetwork that provides the maximum reduction in entropy (uncertainty). The combinatorial search engine 20 identifies data expression patterns in phenotype samples and control samples that exist in one type of sample and not the other to maximize I(F_{S};C) EQ. 1.

I(F_{S};C) is termed combinatorial coordinate dysregulation and is defined as on collective differential expression of the genes in the subnetwork with respect to phenotype. Subnetworks that exhibit combinatorial coordinate dysregulation with respect to a phenotype may shed light into the mechanistic bases of that phenotype. However, identification of such subnetworks is computationally intractable, and due to the combinatorial nature of the associated objective function (I(F_{S};C)), greedy algorithms may not suit well to this problem. This is because, as also demonstrated by the example in FIG. 1, it is not straightforward to bound the combinatorial coordinate dysregulation of a subnetwork in terms of the individual dysregulation of its constituent genes or coordinate dysregulation of its smaller subnetworks (for example, two genes might not be able to discriminate phenotype from control, but addition of a third gene to these two genes might be able to discriminate phenotype from control). Motivated by these considerations, combinatorial coordinate dysregulation of a subnetwork can be decomposed into individual subnetwork state functions and it can be shown that information provided by state functions of larger subnetworks can be bounded using statistics of their smaller subnetworks.

Thus, the objective of the combinatorial search engine 20 is to find subnetwork state functions that are informative of phenotype. For example, Let f_{S}∈{0, 1}^{m} denote an observation of the random variable F_{S}, i.e., a specific combination of the expression states of the genes in S (e.g., if subnetwork S has four genes, f_{S} can be 0011). By definition of mutual information, the combinatorial coordinate dysregulation of S can be written as:

$$\begin{matrix}I ( {F}_{S} ; C ) = {\sum }_{fs ∈ {\{ H , L \}}^{m}} J ( {f}_{s} ; C ) \\ where & EQ . 2 \\ J ( {f}_{s} ; C ) = p ( {f}_{s} ) {\sum }_{c ∈ \{ 0 , 1 \}} p ( c \vert {f}_{s} ) log ( p ( c \vert {f}_{s} ) \text{/} p ( c ) ) & EQ . 3\end{matrix}$$

Here, p(x) denotes P(X=x), that is the probability that random variable X is equal to x (similarly, p(x|y) denotes P(X=x|Y=y)). In biological terms, J(f_{S};C) can be considered a measure of the information provided by subnetwork state function ƒ_{S} (i.e., specific gene expression pattern) on phenotype C. Therefore, a state function ƒ_{S} is informative of phenotype if it satisfies the following conditions:
J(f_{S};C)≧j*, where j* is an adjustable threshold.

−J(f_{S};C)≧J(f_{R};C) for all [symbol] . Here, [symbol] denotes that f_{R} is a substrate of state function ƒ_{S}, that is R⊂S and f_{R} maps each gene in R to an expression level that is identical to the mapping provided by f_{S}. Here, the first condition ensures that the information provided by the state function is considered high enough with respect to a user-defined threshold. It can be shown that for any S⊂V, 0≦J(f_{S};C)≦max{−p(c) log p(c),−(1−p(c)) log(1−p(c))}=jmax(p(c)), where p(c) denotes the fraction of phenotype samples among all available samples. Therefore, in practice, the user specifies a threshold j** in the range [0, 1] and the algorithm adjusts it as j*=j**jmax(p(c)), to make the scoring criterion interpretable and uniform across all datasets. The second condition ensures that informative state functions are non-redundant, that is, a state function is considered informative only if it provides more information on the phenotype than any of its substates. This restriction ensures that the expression of each gene in the subnetwork provides additional information on the phenotype, capturing the synergy between multiple genes to a certain extent. For a given set of phenotype and control samples and a reference PPI network, the objective of the framework is to identify all informative state functions.

Since the space of state functions is large, the problem of discovering all informative state functions can be intractable. This can be addressed by utilizing a bound on the value of J to effectively prune the search space. The information that can be provided by all superstates of a given state function can be bounded based on statistics of that state function, without any information about the superstate. For example, let a subnetwork S⊂V and associated state function ƒ_{S}. For any f_{R} [symbol] f_{S}, the following bound holds:

$$f ( {f}_{R} ; C ) \leq p ( {f}_{s} ) {max}_{c ∈ \{ 0 , 1 \}} \{ p ( c \vert {f}_{S} ) log \frac{1}{p ( c )} \} = {J}_{bound} ( {f}_{S} , C )$$

This theorem does not state that the J-value of a state function is bounded by the J-value of its smaller parts, it rather provides a bound on the J-value of the larger state function based on simpler statistics of its smaller parts.

Using this bound, an algorithm for the identification of combinatorially dysregulated subnetworks (hereinafter, referred to as “CRANE”) was developed to efficiently search for informative state functions. CRANE enumerates state functions in a bottom-up fashion, by pruning out the search space effectively based on the following principles:
1. A state function ƒ_{S} is said to be a candidate state function if |S|=1 or J(ƒ_{S};C)≧J(ƒ_{S}\{g};C) for all gᵢ∈S.
2. A candidate state function ƒ_{S} is said to be extensible if ^{J}bound(ƒ_{S};C)≧j*. This restriction enables pruning of larger state functions using statistics of smaller state functions.
3. An extension of state function ƒ_{S} is obtained by adding one of the H or L states of a gene gᵢ∈ [symbol] \S such that gᵢgⱼ∈ε, where gᵢ is the most recently added gene to fS. This ensures network connectivity of the subnetwork associated with the generated state functions.
4. For an extensible state function, all possible extensions are considered and among those that qualify as candidate state functions, the top b state functions with maximum J(•) are selected as candidate state functions. Here, b is an adjustable parameter that determines the breadth of the search and the case b=1 corresponds to a greedy algorithm.

5. An extensible state function ƒ_{S} is not extended if |S|=d. Here, d is an adjustable parameter that determines the depth of the search.

CRANE enumerates all candidate state functions that qualify according to these principles, for given j*, b, and d. At the end of the search process, the candidate state functions that are not superceded by another candidate state function (the leaves of the enumeration tree) are identified as informative state functions, if their J-value exceeds j*. A detailed pseudo-code for this procedure is given as Algorithm 1.

Algorithm 1:

CRANE-Extend State Function ((S, ƒ_{S}), T, j*, b, d)h: Extends a subnetwork and associated state function. Invoked for each gᵢ∈V and Êᵢ∈{0,1} as CRANE-EXTENDSTATEFUNCTION, (({gᵢ}, {Êᵢ}), Ø, j*, b, d), where j*, b, and d are user-defined.

### Global:

V: Set of genes: C: Phenotype vector
E: Gene expression associated with V and C, ε: PPI dataset associated with V

### Inputs:

(S, ƒ_{S}): Subnetwork/state-function pair to be extended
j*: Threshold on information provided by a state function on phenotype
d: Maximum number of genes in a subnetwork
b: Maximum number of immediate extensions of a subnetwork/state-function pair

### Input/Output:

[symbol] : Set of state functions informative of phenotype

1: if |S| = = d then
2: if (J(fₛ;C) ≧ j* then
3: [symbol] ← [symbol] ∪ {(S, fₛ)}
4: end if
5: return
6: end if
7: [symbol] ← Ø; gᵢ ← most recently added gene to S
8:for each gₖ : (gᵢ,gₖ) ε [symbol] and gₖ ∉S do
9: S′← S ∪ {gₖ}
10: for each Ê_{κ}ε {0,1} do
11: fs′ ← fs′ ∪ Ê_{κ}; redundant ←false
12: for gj ε S′ do
13: if (J(f_{s′\[gi]};C) ≧ J(f_{s′};C) then
14: redundant ← true; break
15: end if
16: end for
17: if (not redundant) and ((Jₘₐₓ((f_{s′};C) > j*)) then
18: [symbol] ← [symbol] ∪ {(S′,f_{s′})}
19: end if
20: end for
21: end for
22: if [symbol] = Ø then
23: if J(fₛ;C) ≧ j* then
24: [symbol] ← [symbol] ∪ {((S, fₛ)}
25: end if
26: return
27: end if
28: [symbol] ← set of top b subnetwork/state-function pairs in Q with respect
to J(f_{s′};C)
29: for each ((S′, f_{s′}) ε [symbol] do
30: CRANE-EXTENDEDSTATEFUNCTION ((S′, f_{s′}), [symbol] , j*, b, d)
31: end for

By way of example, a combinatorial coordinately dysregulated biomarker subnetwork for determining metastasis of colorectal cancer (CRC) can be identified using CRANE. The combinatorial coordinately dysregulated biomarker subnetwork identified by CRANE includes the gene expression profiles of at least two of TNFSF11, MMP1, BCAN, MMP2, thrombospondin 1 (TBSH1), or (osteopontin) SPP1. In an aspect of the application, the state function of TNFSF11, MMP1, BCAN, MMP2, TBSH1, and SPP1 indicative of metastasis of CRC are, respectively, low gene expression (L), low gene expression (L), low gene expression (L), low gene expression (L), and high gene expression (H) (i.e., LLLLLH).

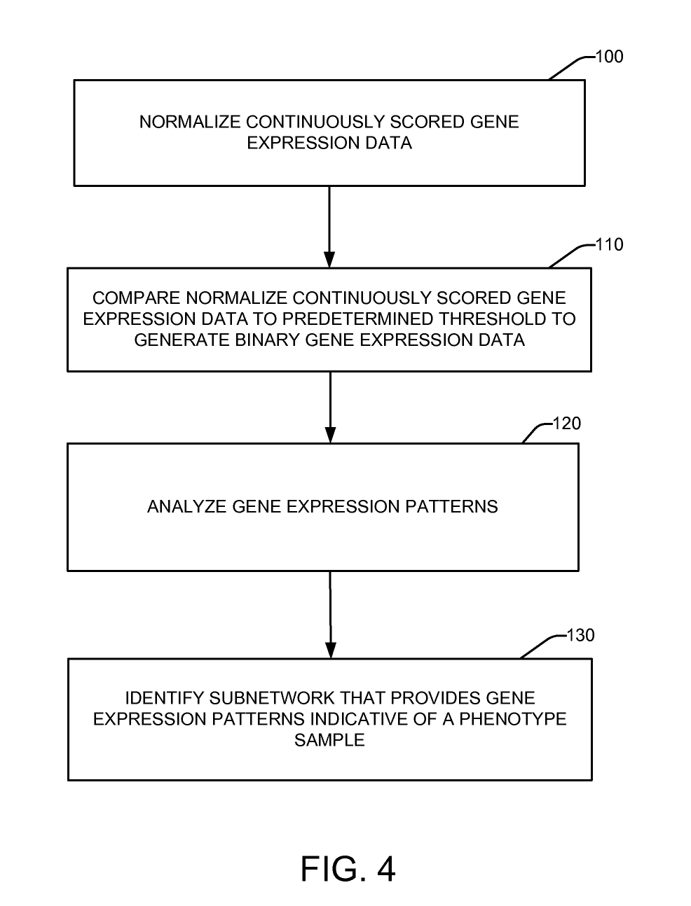

In view of the foregoing structural and functional features described above, the methodology will be better appreciated with reference to FIG. 4. It is to be understood and appreciated that the illustrated actions, in other embodiments, may occur in different orders and/or concurrently with other actions. Moreover, not all illustrated features may be required to implement a method. It is to be further understood that the following methodology can be implemented in hardware (e.g., a computer or a computer network as one or more integrated circuits or circuit boards containing one or more microprocessors), software (e.g., as executable instructions running on one or more processors of a computer system), or any combination thereof.

FIG. 4 illustrates a methodology for selecting combinatorial coordinately dysregulated biomarker subnetworks in accordance with an aspect of the present invention. At 100, continuously scored gene expression data associated with phenotype samples and control samples is normalized to provide normalized gene expression data. At 110, the normalized gene expression data is compared to a predetermined threshold to provide binary gene expression data associated with phenotype samples and control samples. At 120, subnetwork states of the binary gene expression data associated with phenotype samples and control samples are analyzed to identify gene expression patterns that occur in phenotype samples and do not occur in control samples. At 130, a discriminatory subnetwork is identified that provides gene expression patterns indicative of a sample being a phenotype sample.

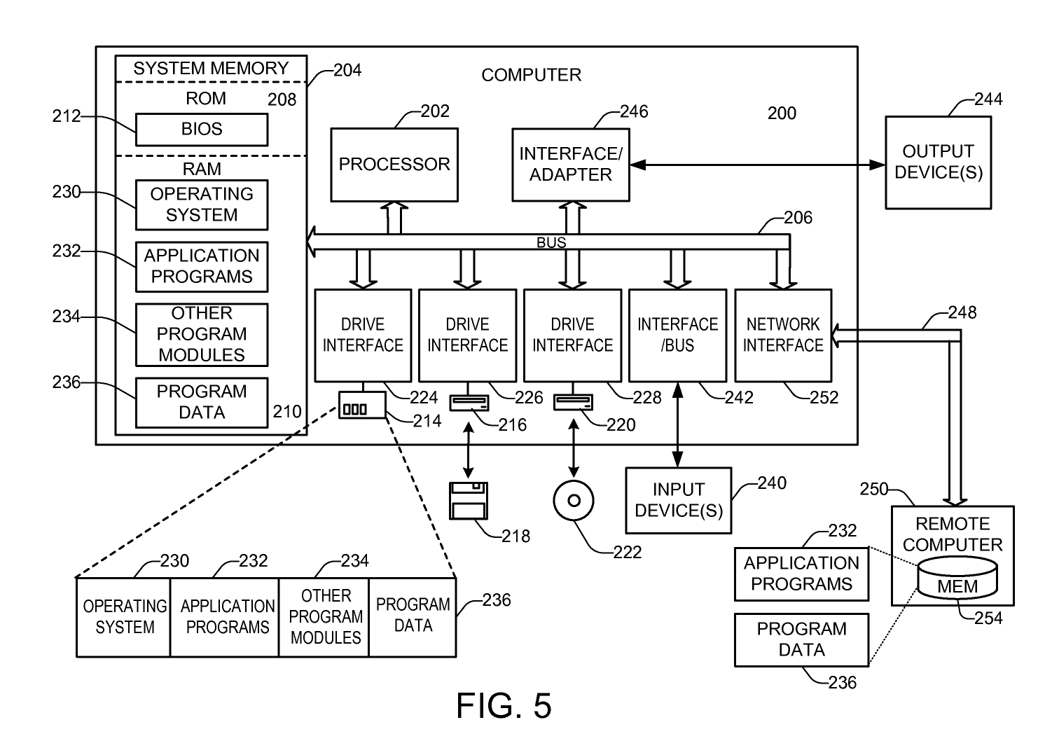

FIG. 5 illustrates a computer system 200 that can be employed to implement systems and methods described herein, such as based on computer executable instructions running on the computer system. The computer system 200 can be implemented on one or more general purpose networked computer systems, embedded computer systems, routers, switches, server devices, client devices, various intermediate devices/nodes and/or stand alone computer systems. Additionally, the computer system 200 can be implemented as part of the computer-aided engineering (CAE) tool running computer executable instructions to perform a method as described herein.

The computer system 200 includes a processor 202 and a system memory 204. A system bus 206 couples various system components, including the system memory 204 to the processor 202. Dual microprocessors and other multi-processor architectures can also be utilized as the processor 202. The system bus 206 can be implemented as any of several types of bus structures, including a memory bus or memory controller, a peripheral bus, and a local bus using any of a variety of bus architectures. The system memory 204 includes read only memory (ROM) 208 and random access memory (RAM) 210. A basic input/output system (BIOS) 212 can reside in the ROM 208, generally containing the basic routines that help to transfer information between elements within the computer system 200, such as a reset or power-up.

The computer system 200 can include a hard disk drive 214, a magnetic disk drive 216, e.g., to read from or write to a removable disk 218, and an optical disk drive 220, e.g., for reading a CD-ROM or DVD disk 222 or to read from or write to other optical media. The hard disk drive 214, magnetic disk drive 216, and optical disk drive 220 are connected to the system bus 206 by a hard disk drive interface 224, a magnetic disk drive interface 226, and an optical drive interface 228, respectively. The drives and their associated computer-readable media provide nonvolatile storage of data, data structures, and computer-executable instructions for the computer system 200. Although the description of computer-readable media above refers to a hard disk, a removable magnetic disk and a CD, other types of media which are readable by a computer, may also be used. For example, computer executable instructions for implementing systems and methods described herein may also be stored in magnetic cassettes, flash memory cards, digital video disks and the like.

A number of program modules may also be stored in one or more of the drives as well as in the RAM 210, including an operating system 230, one or more application programs 232, other program modules 234, and program data 236. The one or more application programs can include the system and methods of selecting combinatorial coordinately dysregulated biomarker subnetworks as previously described in FIGS. 1-4.

A user may enter commands and information into the computer system 200 through user input device 240, such as a keyboard, a pointing device (e.g., a mouse). Other input devices may include a microphone, a joystick, a game pad, a scanner, a touch screen, or the like. These and other input devices are often connected to the processor 202 through a corresponding interface or bus 242 that is coupled to the system bus 206. Such input devices can alternatively be connected to the system bus 206 by other interfaces, such as a parallel port, a serial port or a universal serial bus (USB). One or more output device(s) 244, such as a visual display device or printer, can also be connected to the system bus 206 via an interface or adapter 246. The computer system 200 may operate in a networked environment using logical connections 248 to one or more remote computers 250. The remote computer 250 may be a workstation, a computer system, a router, a peer device or other common network node, and typically includes many or all of the elements described relative to the computer system 200. The logical connections 248 can include a local area network (LAN) and a wide area network (WAN).

When used in a LAN networking environment, the computer system 200 can be connected to a local network through a network interface 252. When used in a WAN networking environment, the computer system 200 can include a modem (not shown), or can be connected to a communications server via a LAN. In a networked environment, application programs 232 and program data 236 depicted relative to the computer system 200, or portions thereof, may be stored in memory 254 of the remote computer 250.

Another aspect of the application relates to the use of expression profiles of the marker proteins described herein in a method for diagnosing an increased risk of development of metastatic colorectal cancer (CRC) in a subject. In one embodiment, the method includes: (1) obtaining a biological sample from a subject comprising colorectal cancer cells; and (2) determining, in the cancer cells, the gene expression level of at least two selected from the selected from the group consisting of TNFSF11, MMP1, BCAN, MMP2, TBSH1, and SPP1, wherein a low level of TNFSF11, MMP1, BCAN, MMP2, and/or TBSH1 and/or high level of SPP1 is indicative of the cancer cells having an increased risk of being metastatic and the subject having metastatic colorectal cancer.

As will be appreciated by those of ordinary skill in the art, sets of biomarkers whose expression profiles correlate with metastatic CRC may be used to identify, study, or characterize unknown biological samples. Accordingly, in one aspect of the present invention, methods for characterizing biological samples obtained from a subject suspected of having metastatic CRC, for diagnosing metastatic CRC in a subject, and for assessing the responsiveness of metastatic CRC in a subject to treatment are contemplated.

The methods of the invention may be applied to the study of any type of biological samples allowing one or more inventive biomarkers to be assayed. Examples of biological samples include, but are not limited to, colon, rectal, bowel, and intestine tissue. In a particular aspect of the present invention, the biological sample is a colon biopsy obtained from the subject.

The biological samples used in the practice of the inventive methods may be fresh or frozen samples collected from a subject, or archival samples with known diagnosis, treatment and/or outcome history. In certain aspects, the inventive methods are performed on the biological sample itself without or with limited processing of the sample.

Preferably, there is enough of the biological sample to accurately and reliably determine the abundance of the set of biomarkers of interest. Multiple biological samples may be taken from the subject in order to obtain a representative sampling from the subject.

In certain aspects, the inventive methods are performed on nucleic acid molecules extracted from cancer cells of the biological sample. For example, RNA may be extracted from the sample before analysis. Methods of RNA extraction are well known in the art (see, for example, J. Sambrook et al., “Molecular Cloning: A Laboratory Manual”, 1989, 2nd Ed., Cold Spring Harbor Laboratory Press: Cold Spring Harbor, N.Y.). Isolated total RNA may then be further purified from the protein contaminants and concentrated by selective ethanol precipitations, phenol/chloroform extractions followed by isopropanol precipitation or cesium chloride, lithium chloride or cesium trifluoroacetate gradient centrifugations. Kits are also available to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Qiagen, Inc. (Valencia, Calif.).

In certain aspects, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each probe being monitored, or using thermostable DNApolymerases (such as avian myeloblastosis virus reverse transcriptase or Moloney murine leukemia virus reverse transcriptase).

The diagnostic methods of the present invention generally involve the determination of the expression levels of a plurality (i.e., one or more, e.g., at least 2, at least 3, at least 4, at least 5 of genes in cancer cells of a biological sample obtained from a subject. Determination of expression levels of nucleic acid molecules in the practice of the inventive methods may be performed by any suitable method, including, but not limited to, Southern analysis, Northern analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds.), 1990, Academic Press: New York), reverse transcriptase PCR(RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320308), one-sided PCR (Ohara et al., Proc. Natl. Acad. Sci., 1989, 86: 5673-5677), in situ hybridization, Taqman based assays (Holland et al., Proc. Natl. Acad. Sci., 1991,88:7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan™, and the like.

Nucleic acid probes for use in the detection of polynucleotide sequences in biological samples may be constructed using conventional methods known in the art. Suitable probes may be based on nucleic acid sequences encoding at least 5 sequential amino acids from regions of nucleic acids encoding a protein marker, and preferably comprise about 15 to about 50 nucleotides. A nucleic acid probe may be labeled with a detectable moiety, as mentioned above in the case of binding agents. The association between the nucleic acid probe and detectable moiety can be covalent or non-covalent. Detectable moieties can be attached directly to nucleic acid probes or indirectly through a linker (E. S. Mansfield et al., Mol. Cell. Probes, 1995, 9: 145-156). Methods for labeling nucleic acid molecules are well-known in the art (for a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994,35:135-153).

Nucleic acid probes may be used in hybridization techniques to detect polynucleotides encoding the biomarkers. The technique generally involves contacting an incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified.

Detection of nucleic acid molecules comprising polynucleotide sequences coding for a protein marker may involve amplification of specific polynucleotide sequences using an amplification method such as PCR, followed by analysis of the amplified molecules using techniques known in the art. Suitable primers can be routinely designed by one skilled in the art. In order to maximize hybridization under assay conditions, primers and probes employed in the methods of the invention generally have at least 60%, preferably at least 75% and more preferably at least 90% identity to a portion of nucleic acids encoding a protein marker.

Hybridization and amplification techniques described herein may be used to assay qualitative and quantitative aspects of expression of nucleic acid molecules comprising polynucleotide sequences coding for the inventive protein markers.

Alternatively, oligonucleotides or longer fragments derived from nucleic acids encoding each protein marker may be used as targets in a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state level of large numbers of polynucleotide sequences simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; Chen et al., Genomics, 1998, 51: 313324; U.S. Pat. Nos. 5,143,854; 5,445,934; 5,807,522; 5,837,832; 6,040,138; 6,045,996; 6,284,460; and 6,607,885).

Once the levels of the biomarkers of interest have been determined for the biological sample being analyzed, they are compared to the levels to at least one expression profile map based on a combinatorial coordinately dysregulated biomarker subnetworks for metastasis of colorectal cancer (CRC), such as described above. Comparison of levels according to methods of the present invention is preferably performed after the levels obtained have been corrected for both differences in the amount of sample assayed and variability in the quality of the sample used (e.g., amount and quality of mRNA tested). Correction may be carried out using different methods well-known in the art. In case of samples containing nucleic acid molecules, correction may be carried out by normalizing the levels against reference genes (e.g., housekeeping genes) in the same sample. Alternatively or additionally, normalization can be based on the mean or median signal (e.g., Ct in the case of RT-PCR) of all assayed genes or a large subset thereof (global normalization approach).

Using methods described herein, skilled physicians may select and prescribe treatments adapted to each individual subject based on the diagnosis of metastatic CRC provided to the subject through determination of the levels of the inventive biomarkers. In particular, the present invention provides physicians with a non-subjective means to diagnose metastatic CRC, which will allow for early treatment, when intervention is likely to have its greatest effect. Selection of an appropriate therapeutic regimen for a given patient may be made based solely on the diagnosis provided by the inventive methods. Alternatively, the physician may also consider other clinical or pathological parameters used in existing methods to diagnose CRC and assess its advancement.

### Example

In this Example, we evaluated the performance of CRANE in identifying state functions associated with metastasis of colorectal cancer (CRC). We first compared the classification performance of the subnetworks associated with these state functions against single gene markers and subnetworks identified by an algorithm that aims to maximize additive coordinate dysregulation. We then presented comprehensive experimental results to evaluate the effect of parameters on the performance of CRANE. Subsequently, with a view to investigating the benefits of pruning the subnetwork search space, we compared CRANE′S performance with a version that does not use the bound on J(•) value to prune the search space. Finally, we inspected the subnetworks that are useful in classification, and discuss the insights these subnetworks can provide into the metastasis of CRC.

### Datasets

In our experiments, we use two CRC related microarray datasets obtained from GEO (Gene Expression Omnibus, http://www.ncbi.nlm.nih.gov/geo/index.cgi). These datasets, referenced by their accession number in the GEO database, include the following relevant data:
GSE6988 contains expression profiles of 17,104 genes across 29 vs. 51 colorectal tumor samples with and without liver metastasis.
GSE3964 contains expression profiles of 5,845 genes across 28 vs. 18 colorectal tumor samples with and without liver metastasis.

The human protein-protein interaction data used in our experiments is obtained from the Human Protein Reference Database (HPRD, http://www.hprd.org). This dataset contains 35023 binary interactions among 9299 proteins, as well as 1060 protein complexes consisting of 2146 proteins. We integrate the binary interactions and protein complexes using a matrix model (e.g., each complex is represented as a clique of the proteins in the complex), to obtain a PPI network composed of 42781 binary interactions among 9442 proteins.

### Experimental Design

For each of the datasets mentioned above, we discover informative state functions (in terms of discriminating tumor samples with or without metastasis) using CRANE. While state functions that are indicative of either metastatic or non-metastatic phenotype can have high J(•) values, we use only those that are indicative of (i.e., knowledge of which increases the likelihood of) metastatic phenotype for classification and further analyses, since such state functions are directly interpretable in terms of their association with metastasis.

In the experiments reported here, we set b=10. d is set at 3 for GSE3964 and at 6 for GSE6988. The value of j** is set to and 0.15 and 0.40 for discovery of subnetworks on GSE3964 and GSE6988 respectively. The top five non-overlapping subnetworks discovered on GSE6988 by CRANE using these parameter settings are shown in Table 1. Note that these parameters are used to balance the trade-off between computational cost of subnetwork identification and classification accuracy. The reported values are those that provide reasonable performance by spending a reasonable amount of time on subnetwork identification (a few hours in Matlab for each dataset). The effect of different values of these parameters on CRANE′S performance are presented later in this section.

To binarize the gene expression datasets, we first normalized the gene expression profiles so that each gene has an average expression of 0 and standard deviation 1. Then we set the top a fraction of the entries in the normalized gene expression matrix to H (high expression) and the rest to L (low expression). In the reported experiments, we use α=0.25 (25% of the genes are expressed on an average) as this value is found to optimize the classification performance.

TABLE 1
Five Non-Overlapping subnetworks that are associated with the
most informative State Functions Discovered on GSE6988 with
D = 6 and the functional Enrichment of these subnetworks

| | | Comb. | Most | |
|---|---|---|---|---|
| | | Coor. | Significantly | Enrich- |
| | | Dysreg- | Enriched | ment |
| Rank | Proteins | ulation | Process | p-value |
| 1 | SERPINA3, KLK3, EPOR, | 0.94 | Inflammation | 1 × 10⁻³ |
| | GNB2L1, RASA1, RAF1 | | | |
| 2 | E2F4, CCNE1, GSK3B, | 0.85 | Cell movement | 1 × 10⁻³ |
| | HNRPD, SF3B2, RPL13 | | | |
| 3 | DMTF1, CCND2, AKAP8, | 0.885 | Cell migration | 1 × 10⁻⁴ |
| | DDX5, FN1, CRP | | | |
| 4 | ANXA11, PLSCR1, | 0.81 | Cell Adhesion | 1 × 10⁻⁴ |
| | ESWR1, PTK2B, ITGB2, | | | |
| | HP | | | |
| 5 | SKP1A, CCNA2, | 0.81 | Inflammation | 1 × 10⁻⁴ |
| | CDKN1A, GADD45G, | | | |
| | EEF1G, RGL2 | | | |

### Implementation of Other Algorithms

We identified single gene markers by running CRANE with d=1 (i.e., by searching for subnetworks composed of one gene). We also identified coordinately dysregulated subnetworks using an additive algorithm, that is an algorithm that aims to maximize additive coordinate dysregulation. The additive algorithm identified a subnetwork associated with each gene in the network by seeding a greedy search process from that gene. It grows subnetworks by iteratively adding to the subnetwork a network neighbor of the genes that are already in the subnetwork. At each iteration, the neighbor that maximizes the coordinate dysregulation of the subnetwork is selected to be added. Once all subnetworks were identified, we sorted these subnetworks according to their coordinate dysregulation (I(E_{S}; C) or (I(F_{S}; C) and use the top K disjoint subnetworks to train and test classifiers, for different values of K. While quantizing E_{S} to compute (I(E_{S}; C), we used [log₂ (|U|]+1 bins where |U| denotes the number of samples. The subnetworks identified by the greedy algorithm are filtered through three statistical tests. In our experiments, these statistical tests are not performed for the subnetworks discovered by the additive algorithm and CRANE. This is because, testing of statistical significance based on multiple runs on permuted instances is computationally expensive, since CRANE performs an almost exhaustive search of the subnetwork space. In this respect, development of efficient algorithms for testing statistical significance of subnetworks identified by such exhaustive algorithms remains an important problem.

For the subnetworks with additive coordinate dysregulation, we computed the subnetwork activity ES for each subnetwork, and use these as features to train and test two different classifiers: (i) a SVM using Matlab's svmtrain and svmclassify functions (this method is not applicable to combinatorial coordinate dysregulation), (ii) feed-forward neural networks, in which each input represents the subnetwork activity for a subnetwork and these inputs are connected to hidden layer neurons. For the single-gene markers, we ranked all genes according to the mutual information of their expression profile with phenotype (I(Eᵢ; C)) and use the expression level of K genes with maximum I(Eᵢ; C) as features for classification.

### Classification Performance

We evaluated the cross-classification performance of the subnetworks in the context of predicting metastasis of CRC. Namely, we use subnetworks discovered on the GSE6988 dataset to train classifiers and we test the resulting classifiers on GSE3964. Similarly, we use subnetworks discovered on GSE3964 to train classifiers using the same dataset and perform testing of these classifiers on 28 metastatic and 20 randomly selected non-metastatic samples on GSE6988. The cross-classification performance of subnetworks discovered by an algorithm is not only indicative of the power of the algorithm in discovering subnetworks that are descriptive of phenotype, but also the reproducibility of these subnetworks across different datasets.

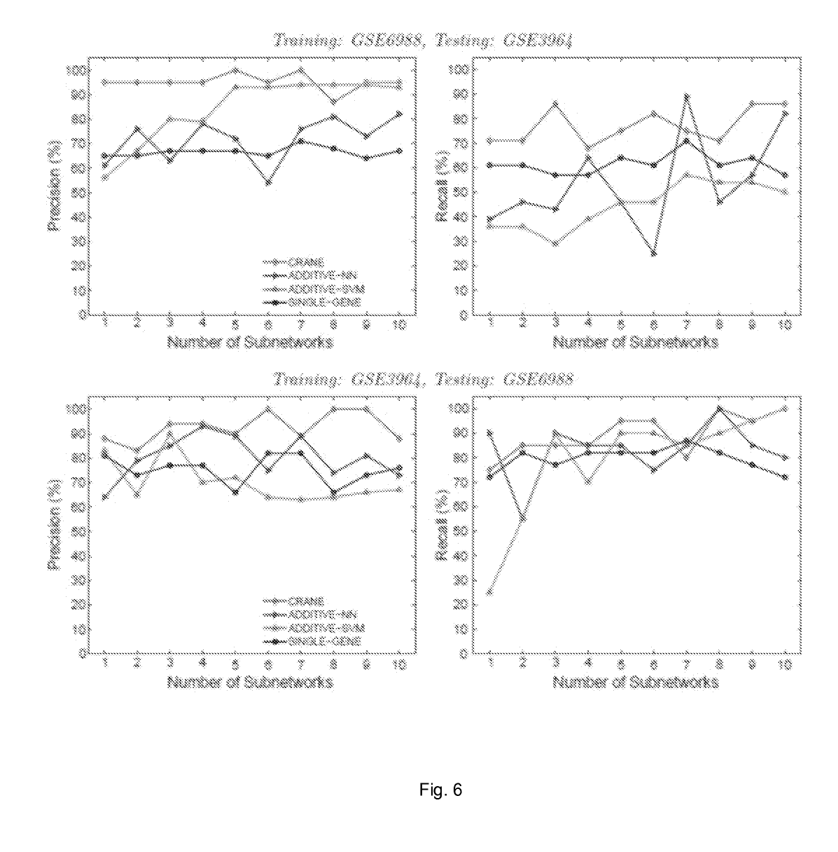

The classification performance of the subnetworks identified by CRANE, the additive algorithm, and single gene markers are compared in FIG. 6. In FIG. 6, for each 1≦K≦10, the precision and recall achieved by each classifier are reported. These performances criteria are defined as follows:

$$\begin{matrix}Precision = \begin{matrix}\# true positives \\ \# true positive + \# false positives\end{matrix} & ( 25 ) \\ recall = \frac{\# true positives}{\# true positives + \# false negatives} & ( 26 )\end{matrix}$$

Here, a true positive is defined as a metastatic sample that is correctly predicted as a metastatic sample, while a false positive is a non-metastatic sample that is incorrectly predicted as metastatic. A false negative is a metastatic sample that is incorrectly predicted as non-metastatic. Therefore, precision quantifies the fraction of true positives among all samples predicted as metastatic by the classifier, while recall quantifies the fraction of true positives among all metastatic samples.

As seen in FIG. 6, subnetworks identified by CRANE outperform the subnetworks identified by other algorithms in predicting metastasis of colorectal cancer. In fact, in both cases, CRANE has the potential to deliver very high accuracy using very few subnetworks (maximum precision of 100% on both GSE6988 and GSE3964, maximum recall of and 95% and 86% for classification of samples in GSE6988 and GSE3964, respectively). While we use a simple feature selection method here for purposes of illustration, the performance of CRANE subnetworks are quite consistent, suggesting that these performance figures can indeed be achieved by developing elegant methods for selection of subnetwork features. These results are rather impressive, given that the best performance that can be achieved by the additive algorithm is 82%/93% precision and 89%/100% recall for the classification of GSE3964 and GSE6988, respectively. Note that, while the performance of other algorithms is improved by increasing number of subnetwork features, the performance of CRANE appears to decline. This is likely because CRANE represents subnetwork features as multi-dimensional state functions. Therefore, while a few subnetworks each containing a few genes provide sufficient information for accurate classification, the accuracy declines as more subnetworks are incorporated because of the growth in dimensionality.

### Effect of Pruning

An important feature of CRANE is the use of a theoretical bound on J(•) to prune out the search space. In order to verify the effectiveness of this feature in improving the efficiency of CRANE, as well as its ability to discover informative subnetworks, we compare CRANE with a version that does not apply pruning using the bound on J(•). These experiments are performed on GSE6988, by fixing b=10, j**=0.45, α=0.25, and running CRANE and its version without pruning for d ranging from 1 to 8.

The runtimes of the CRANE and the algorithm without pruning were compared. The algorithm without pruning does not scale well with increasing d. This is expected, since the algorithm performs exhaustive search with a breadth of b=10, making the runtime exponential in d. However, by pruning this search space using the bound on J(•), CRANE reduces this runtime drastically, providing orders of magnitude improvement for larger values of d. Note that, if b=∞ both CRANE and its version without pruning are guaranteed to discover all subnetworks with J(•) j*. However, since the breadth of search is limited by parameter b, both algorithms may miss some subnetworks. CRANE is able to identify all subnetworks that are identified by the version without pruning; i.e., CRANE achieves the drastic improvement in runtime without compromising sensitivity. These results clearly demonstrate the value of using the theoretical bound on J(•) value while searching for informative subnetworks.

### Effect of Parameters

We also investigated the effect of parameters used to configure CRANE on classification performance of identified subnetworks, by fixing all but one of the parameters to the above-mentioned values and varying the remaining parameter. The tuneable parameters of CRANE are the following:

d: d is the maximum size of a subnetwork. CRANE stops extending a subnetwork when the number of genes in the subnetwork reaches d. In other words, d determines the depth of the search.

b: b is the number of state functions selected by CRANE at each iteration with maximum J(•) value. Thus, b determines the breadth of the search.

j**: j** is the minimum J(•) value of a subnetwork state function to be considered informative.

α: α is the fraction of the entries in the normalized gene expression matrix that is set to H (high expression). The rest of the (1-α) entries of the gene expression matrix is set to L (low expression).

For each configuration of the parameters, we report the average F-measure across different values of the number of subnetworks used in classification, ranging from 1 to 10. Here, F-measure is defined as the harmonic mean of precision and recall, i.e.,

$$F - measure = 2 \times \frac{precision \times recall}{precision + recall} \qquad ( 27 )$$

We observe that classification performance is quite robust against variation in a ranging from 10% to 50%, while best performance is observed at α=25%. As expected, classification performance improves by increasing j**. Increasing the breadth of search (b) improves classification performance in general, which is also expected since larger values of b enable exploration of the search space further. Note that the special case with b=1 is algorithmically equivalent to the additive algorithm with a different objective function (combinatorial coordinate dysregulation as opposed to additive coordinate dysregulation). We observe that CRANE outperforms the additive algorithm with b=1 as well, indicating that the combinatorial formulation of coordinate dysregulation is potentially more useful than the additive formulation for classification.

Increasing d improves performance as would be expected; however this improvement saturates for d>3 and performance declines for larger subnetworks. This observation can be attributed to curse of dimensionality, since the number of possible values of random variable F (expression state of a subnetwork) grows exponentially with increasing subnetwork size. We also investigated the effect of parameter d on CRANE's ability to discover larger subnetworks. For this purpose, we compared the subnetworks identified by CRANE on GSE6988 using d=7 and d=8 with those identified using d=6. The top five non-overlapping subnetworks identified using d=7 and d=8 are shown in Table 2. Comparison of the subnetworks in Tables 1 and 2 shows that, while there is some overlap in subnetworks discovered using different values of d, some subnetworks that can be discovered for larger values of d can be missed if a smaller value of d is used. Note, however, that this does not mean that smaller subnetworks of these subnetworks are not discovered by CRANE. Rather, such subnetworks are often eliminated because of their overlap with subnetworks that have higher combinatorial coordinate dysregulation. Indeed, comprehensive comparison of subnetworks shows that many of the subnetworks composed of seven genes, which are discovered using d=7, are identified as different six-gene combinations when d is set 6. In other words, if d is set to a smaller value, then a larger “naturally occurring” subnetwork can be “truncated” into smaller subnetworks. For this reason, the parameter d needs to be set carefully, possibly by using different values of d and inspecting the size and gene content of subnetworks discovered for each d.

TABLE 2
Five Non-Overlapping Subnetworks that Are Associated
with the Most Informative State Functions Discovered
on GSE6988 for d = 7 and d = 8

| | | Combinatorial |
|---|---|---|
| | | Coordinate |
| Rank | Proteins | Dysregulation |
| d = 7 | | |
| 1 | MYC, GJB5, RPL35A, TRPM1, | 0.81 |
| | HYOU1, ARK3, CDK10 | |
| 2 | FLJ20487, ATP5F1, GNG3, | 0.79 |
| | RBM17, RCN1, ENO3 | |
| 3 | TCF7, NDUFV2, ARMCX1, | 0.79 |
| | LRP16, PPT1, KIF13B, | |
| | MSL3L1 | |
| 4 | SMARCA4, ATP5D, BTBD2, | 0.76 |
| | FGFBP1, PPP2R5D, PYCARD, | |
| | PPP2R3A | |
| 5 | MPG, NT5C2, RCL1, | 0.76 |
| | SH3BP5, MYT1, EEF1G | |
| d = 8 | | |
| 1 | RARS, ARMCX1, SDF2, | 0.92 |
| | ARF3, HYOU1, F11R, | |
| | LOC284361, ENO3 | |
| 2 | C18orf10, ATP5F1, VAV1, | 0.89 |
| | FBXW11, ASRGL1, EFHD1, | |
| | KIAA0182, NDUFA1 | |
| 3 | SURB7, BRCA1, EXOSC4, | 0.78 |
| | PCBP2, RAD51C, TCF7, | |
| | BCL2A1, CREB3 | |
| 4 | BMX, RAD51L3, PIN1, | 0.77 |
| | SLAMF1, CKLF, ELA3A, | |
| | MMP2, N-PAC | |
| 5 | SMARCA4, ATP5D, BTBD2, | 0.76 |
| | FGBP1, PPP2R5D, PYCARD, | |
| | PPP2R3A | |

### Subnetworks and State Functions Indicative of Metastasis in CRC

Cancer metastasis involves the rapid proliferation and invasion of malignant cells into the bloodstream or lymphatic system. The process is driven, in part, by the dysregulation of proteins involved in cell adhesion and motility, the degradation of the extracellular matrix (ECM) at the invasive front of the primary tumor, and is associated with chronic inflammation. An enrichment analysis of the top five subnetworks identified on GSE6988 reveals that all of these subnetworks are highly significant for the network processes underlying these phenotypes (Table 1).

Further, as CRC metastasis is our classification endpoint, we wanted to evaluate our subnetworks in terms of their potential to propose testable hypotheses. In particular, to highlight the power of our model approach, we chose a subnetwork for which at least one gene was expressed in the state function indicative of CRC metastasis. This subnetwork contains TNFSF11, MMP1, B CAN, MMP2, TBSH1, and SPP1 and the state function LLLLLH (in respective order) indicates metastatic phenotype with J-value 0.33. The combinatorial dysregulation of this subnetwork is 0.72, while its additive coordinate dysregulation is 0.37, i.e., this is a subnetwork which would likely have escaped detection by the additive algorithm (this subnetwork is not listed in Table 1 since it is not among the top five scoring subnetworks). Using the genes in this subnetwork as a seed, we constructed a small subnetwork diagram for the purpose of more closely analyzing the post-translational interactions involving these proteins. This is done using Metacore, a commercial platform that provides curated, highly reliable interactions. From this subnetwork, we removed all genes indicated to be not expressed in human colon by the database, and then selectively prune it in order to clearly focus on a particular set of interactions. It merits noting that, although Brevican (BCAN) is in subnetwork, it is removed for being non-expressed in the human colon, although evidence from the Gene Expression Omnibus (see accession GDS2609) casts doubt on this, as does the microarray we use for scoring (GSE6988).

SPP1 (osteopontin) and TBSH1 (thrombosponidin 1) interacted with a number of the integrin heterodimers to increase their activity. Integrin heterodimers play a major role in mediating cell adhesion and cell motility. SPP1, up-regulated in metastasis, is a well-studied protein that triggers intracellular signaling cascades upon binding with various integrin heterodimers, promotes cell migration when it binds CD44, and when binding the alpha-5/beta-3 dimer in particular, promotes angiogenesis, which is associated with the metastatic phenotype of many cancers. MMP proteins are involved in the breakdown of ECM, particularly collagen which is the primary substrate at the invasive edge of colorectal tumors. MMP-1 has an inhibitory effect on vitronectin, hence the loss of expression of MMP-1 may “release the brake” on vitronectin, which in turn may increase the activity of the alpha-v/beta-5 integrin heterodimer. Likewise, MMP-2 shows an inhibitory interaction with the alpha-5/beta-3 dimer, which may counteract to some extent the activating potential of SPP1, suggesting that a loss of MMP-2 may exacerbate the metastatic phenotype. Taken together, these interactions suggest a number of perturbation experiments, perhaps by pharmacological inhibition or siRNA interference of the integrin dimmers or MMP proteins, to evaluate the role of these interactions, individually or synergistically, in maintaining the metastatic phenotype. Note also that, alpha-v/beta-5 integrin does not exhibit significant differential expression at the mRNA-level, suggesting that the state function identified by CRANE may be a signature of its post-translational dysregulation in metastatic cells.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the scope of the appended claims.

1. A system for selecting combinatorial coordinately dysregulated biomarker subnetworks, the system comprising:
memory for storing computer executable instructions;
a processor for accessing memory and executing computer executable instructions, the computer executable instructions comprising;
a gene data binarization component that compares continuous scored gene expression data associated with phenotype samples and control samples to a predetermined threshold to provide binary gene expression data associated with phenotype samples and control samples; and
a combinatorial search engine that analyzes subnetwork states of binary gene expression data associated with phenotype samples and control samples to identify gene expression patterns that occur in phenotype samples and do not occur in control samples to identify a subnetwork that provides gene expression patterns indicative of a sample being a phenotype sample.

2. The system of claim 1, the combinatorial search engine identifies gene expression patterns that occur in control samples and do not occur in phenotype samples to identify a subnetwork that provides gene expression patterns indicative of a sample being a normal sample.

3. The system of claim 2, the combinatorial search engine selects a discriminatory biomarker subnetwork that provides gene expression patterns that are in the phenotype sample and not in the normal sample and provides gene expression patterns that are in the normal sample and not in the phenotype sample.

4. The system of claim 1, the computer executable instructions further comprising a gene data normalization component that normalizes scored gene data prior to being provided to the gene data binarization component.

5. The system of claim 1, the combinatorial coordinately dysregulated biomarker subnetwork being indicative of colorectal cancer metastasis.

6. The system of claim 5, the subnetwork including the gene expression profiles of at least two genes of TNFSF11, MMP1, BCAN, MMP2, thrombospondin 1 (TBSH1), or (osteopontin) SPP1.

7. The system of claim 6, state functions of TNFSF11, MMP1, BCAN, MMP2, TBSH1, and SPP1 indicative of metastasis of colorectal cancer being, respectively, low gene expression (L), low gene expression (L), low gene expression (L), low gene expression (L), and high gene expression (H).

8. A method for selecting combinatorial coordinately dysregulated biomarker subnetworks, the method comprising:
normalizing continuously scored gene expression data with phenotype samples and control samples to provide normalized gene expression data;
comparing the normalized gene expression data to a predetermined threshold to provide binary gene expression data associated with phenotype samples and control samples;
analyzing subnetwork states of the binary gene expression data associated with phenotype samples and control samples to identify gene expression patterns that occur in phenotype samples and do not occur in control samples; and
identifying subnetworks that provide gene expression patterns indicative of a sample being a phenotype sample.

9. The method of claim 8, further comprising identifying gene expression patterns that occur in control samples and do not occur in phenotype samples and identifying a subnetwork that provides gene expression patterns indicative of a sample being a normal sample.

10. The method of claim 9, further comprising selecting a discriminatory biomarker subnetwork that provides gene expression patterns that are in the phenotype sample and not in the normal sample and that provides gene expression patterns that are in the normal sample and not in the phenotype sample.

11. The method of claim 10, the combinatorial coordinately dysregulated biomarker subnetwork being indicative of colorectal cancer metastasis.

12. The method of claim 10, the subnetwork including the gene expression profiles of at least two genes of TNFSF11, MMP1, BCAN, MMP2, thrombospondin 1 (TBSH1), or (osteopontin) SPP1.

13. The method of claim 12, state functions of TNFSF11, MMP1, BCAN, MMP2, TBSH1, and SPP1 indicative of metastasis of colorectal cancer being, respectively, low gene expression (L), low gene expression (L), low gene expression (L), low gene expression (L), and high gene expression (H).

14. A combinatorial coordinately dysregulated biomarker subnetwork for determining metastasis of colorectal cancer (CRC), the combinatorial coordinately dysregulated biomarker subnetwork comprising the gene expression profiles of at least two of TNFSF11, MMP1, BCAN, MMP2, thrombospondin 1 (TBSH1), or (osteopontin) SPP1.

15. The combinatorial coordinately dysregulated biomarker subnetwork of claim 14, wherein state function of TNFSF11, MMP1, BCAN, MMP2, TBSH1, and SPP1 indicative of metastasis of CRC being, respectively, low gene expression (L), low gene expression (L), low gene expression (L), low gene expression (L), and high gene expression (H).

16. A method for diagnosing an increased risk of development of metastatic colorectal cancer (CRC) in a subject, the method including: obtaining a biological sample from a subject comprising colorectal cancer cells; and determining, in the cancer cells, the gene expression level of at least two selected from the selected from the group consisting of TNFSF11, MMP1, BCAN, MMP2, TBSH1, and SPP1, wherein a low level of TNFSF11, MMP1, BCAN, MMP2, and/or TBSH1 and/or high level of SPP1 is indicative of the cancer cells having an increased risk of being metastatic and the subject having metastatic colorectal cancer.
